(19) United States
(12) Patent Application Publication
(10) Pub. No.: US 2019/0180882 A1
(43) Pub. Date: Jun. 13, 2019

(54) DEVICE AND METHOD OF PROCESSING MULTI-DIMENSIONAL TIME SERIES MEDICAL DATA

(71) Applicant: Electronics and Telecommunications Research Institute, Daejeon (KR)

(72) Inventors: Youngwoong HAN, Daejeon (KR); Hwin Dol PARK, Daejeon (KR); Myung-eun LIM, Daejeon (KR); Ho-Youl JUNG, Daejeon (KR); Jae Hun CHOI, Daejeon (KR)

(73) Assignee: Electronics and Telecommunications Research Institute, Daejeon (KR)

(21) Appl. No.: 16/031,162

(22) Filed: Jul. 10, 2018

(30) Foreign Application Priority Data
Dec. 12, 2017 (KR) 10-2017-0170715
Apr. 2, 2018 (KR) 10-2018-0038323

(51) Int. Cl. G16H 50/50; G06N 99/00

(52) U.S. Cl. CPC G16H 50/50; G06N 99/005

(57) ABSTRACT

Provided are a device and method for processing multi-dimensional time series medical data. The device for processing multi-dimensional time series medical data according to an embodiment of the present invention includes a network interface, a preprocessing unit, a data analysis unit, and a processor. The network interface may receive time series medical data including first visit data corresponding to the first time and second visit data corresponding to the second time before the first time. The preprocessing unit preprocesses the time series medical data to generate the modeling data. The preprocessing unit is configured to preprocess the first visit data based on a difference between the first time and the second time. The data analysis unit may generate a time series analysis model for predicting future visit data from the modeling data.

### CROSS-REFERENCE TO RELATED APPLICATIONS

This U.S. non-provisional patent application claims priority under 35 U.S.C. § 119 of Korean Patent Application Nos. 10-2017-0170715, filed on Dec. 12, 2017, and 10-2018-0038323, filed on Apr. 2, 2018, the entire contents of which are hereby incorporated by reference.

### BACKGROUND

The present disclosure relates to processing time series data and building a learning model therefor, and more particularly, to a device and method for processing multi-dimensional time series medical data.

The development of various technologies including medical technology improves human standard of living and increases human life span. However, changes in lifestyle and erroneous eating habits due to technological development are causing various diseases. In order to lead a healthy life, there is a need to anticipate the future health condition from treating the current disease.

The development of industrial technology and information and communication technologies is creating a significant amount of information and data. In recent years, technologies such as artificial intelligence that provides various services by learning an electronic device such as a computer using such a large amount of information and data are emerging. Particularly, in order to predict the future health condition, a method of constructing a learning model using various medical data or health data has been proposed. Medical data differs from data collected in other fields, for example, depending on features such as typicalness, scarcity, or non-uniformity. Thus, there is a need for effective treatment of medical data to predict future health conditions.

### SUMMARY

The present disclosure is to provide a device and method for processing multi-dimensional time series medical data so as to secure reliability, accuracy, and efficiency of future health condition prediction based on the complex characteristics of a human being.

An embodiment of the inventive concept provides a device for processing multi-dimensional time series medical data according to an embodiment of the inventive concept includes a network interface, a preprocessing unit, a data analysis unit, and a processor. The network interface may receive time series medical data including first visit data corresponding to the first time and second visit data corresponding to the second time before the first time. The preprocessing unit preprocesses the series medical data to generate the modeling data. The data analysis unit may generate a time series analysis model for predicting future visit data from the modeling data. The processor controls the preprocessing unit and the data analysis unit.

For example, the preprocessing unit may preprocess the first visit data based on the difference between the first time and the second time. For example, the modeling data may include first modeling visit data obtained by preprocessing the first visit data, and second modeling visit data obtained by preprocessing the second visit data, and the first modeling visit data may include time-gap data generated based on a difference between the first time and the second time.

For example, the first visit data may include first feature data, which is numerical data, and second feature data, which is non-numeric data. The processor may convert the second feature data into numerical data. The preprocessing unit normalizes the first feature data to have a numerical value in the reference range, converts the non-numeric data of the second feature data into binary data, and converts the binary data into numerical data having numerical values in the reference range.

In one example, the preprocessing unit may generate the first masking data and the second masking data. The first masking data may have a first data value if target feature data exist in the first visit data and a second data value if the target feature data does not exist in the first visit data. The second masking data may have a first data value if target feature data exists in the second visit data and a second data value if target feature data does not exist in the second visit data. The preprocessing unit may generate the first modeling visit data by preprocessing the first visit data and the first masking data, and the second modeling visit data by preprocessing the second visit data and the second masking data.

In an embodiment of the inventive concept, a method for processing multi-dimensional time series medical data by a processor includes: preprocessing a first visit data including a plurality of feature data extracted during a first time and a second visit data including a plurality of feature data extracted during a second time before the first time; and learning a time series analysis model for predicting future visit data including a plurality of feature data based on the preprocessed first and second visit data. For example, the preprocessing of the first visit data and the second visit data may include preprocessing the first visit data by reflecting the time-gap data corresponding in the difference between the first time and the second time to the first visit data.

For example, the preprocessing of the first visit data and the second visit data may further include learning an encoding model for changing a dimension of each of the first and second visit data to a reference dimension based on the first and second visit data. Personal time series medical data may be preprocessed based on the learned encoding model and personal future visit data may be predicted based on the preprocessed personal time series medical data and the learned time series analysis model.

For example, the preprocessing of the first visit data and the second visit data may further include adding first masking data to the first visit data and adding second masking data having the same dimension as the first masking data to the second visit data. The encoding model may be learned based on the first and second visit data and the first and second masking data.

For example, the preprocessing of the first visit data and the second visit data may include learning the numerical model based on the non-numeric data included in the first and second visit data. The preprocessing of the first visit data and the second visit data may include normalizing the numerical data included in the first and second visit data, and learning the encoding model based on the normalized or converted first and second visit data.

### BRIEF DESCRIPTION OF THE FIGURES

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a view illustrating a health condition prediction system according to an embodiment of an inventive concept;

FIG. 2 is an exemplary block diagram of the time series medical data processing device of FIG. 1;

FIG. 3 is a view for explaining time series medical data processed by the time series medical data processing device of FIG. 1;

FIG. 4 is a view for explaining a data processing process of the time series medical data processing device of FIG. 1;

FIG. 5 is a view for explaining a preprocessing process in the method of processing time series medical data of FIG. 4; and

FIG. 6 is a view for explaining an application process of masking data in the method of processing time series medical data of FIG. 4.

### DETAILED DESCRIPTION

In the following, embodiments of the inventive concept will be described in detail so that those skilled in the art easily carry out the inventive concept.

FIG. 1 is a view illustrating a health condition prediction system according to an embodiment of an inventive concept. Referring to FIG. 1, a health condition prediction system 100 includes a terminal 110, a medical database 120, a time series medical data processing device 130, a preprocessing model database 140, a prediction model database 150, and a network 160.

The terminal 110 collects the time series medical data from the user and provides the collected data to the time series medical data processing device 130. The time series medical data may refer to data representing a health condition of a user generated by diagnosis, treatment, or medication prescription at a medical institution, such as Electronic Medical Record (EMR) data. The time series medical data may include visit data generated when visiting a medical facility for diagnosis, treatment, or medication prescription. Such visit data may be generated each time a visit may be made to a medical institution, and a plurality of visit data listed in a time series may be included in the time series medical data. Each of the plurality of visit data may include a plurality of feature data generated based on diagnostic, therapeutic, or medication-prescribed features. For example, the feature data may be data measured by a test such as blood pressure or data representing the degree of a disease such as atherosclerosis.

The terminal 110 may be one of various electronic devices capable of receiving time series medical data from a user such as a smart phone, a desktop, a laptop, and a wearable device. The terminal 110 may include a communication module or a network interface to transmit time series medical data via the network 160. FIG. 1 illustrates one terminal 110, but is not limited thereto. Time series medical data may be provided to a time series medical data processing device from a plurality of terminals.

The medical database 120 is configured such that medical data for various users are managed in an integrated manner. For example, the medical database 120 may receive medical data from public institutions, hospitals, and users. The medical database 120 may be implemented in a server or storage medium. The medical data may be managed in a time series in the medical database 120, and may be grouped and stored. The medical database 120 may periodically provide time series medical data to the time series medical data processing device 130 via the network 160.

The time series medical data processing device 130 may construct a learning model through time series medical data received from the medical database 120 (or the terminal 110). For example, a learning model may include a preprocessing model for preprocessing time series medical data or a prediction model for predicting future health conditions based on preprocessed time series data. The time series medical data processing device 130 may learn the time series medical data received from the medical database 120 to generate a learning model.

The time series medical data processing device 130 may process the time series medical data received from the terminal 110 based on the constructed learning model. The time series medical data processing device 130 may preprocess time series medical data based on the pre-processing model constructed according to the learning result. Also, the time series medical data processing device 130 may analyze the preprocessed time series medical data based on the prediction model constructed according to the learning result. As a result of analysis, the time series medical data processing device 130 may calculate the medical data (visit data) for the future time.

The time series medical data processing device 130 may predict the future health condition of the user based on the calculated medical data (visit data) The predicted future health condition may be provided to the terminal 110 via the network 160 at the request of the terminal 110. However, the inventive concept is not limited thereto. The time series medical data processing device 130 predicts future visit data based on the constructed learning model and predicts a future health condition of the user in a separate electronic device. For example, a separate electronic device may be the terminal 110, and the time series medical data processing device 130 may transmit future visit data to the terminal 110 via the network 160.

The preprocessing model database 140 is configured so that the preprocessing models generated by learning in the time series medical data processing device 130 are integratedly managed. The preprocessing model database 140 may be implemented in a separate server or storage medium. However, the inventive concept is not limited thereto. The preprocessing model may be managed by a processor in the time series medical data processing device 130 and may be stored in a storage of the time series medical data processing device 130 or the like. The preprocessing model may include a digitization model for digitizing the time series medical data and an encoding model for changing the dimension of the time series medical data to a fixed dimension. Specific examples of such a preprocessing model will be described later.

The prediction mode database 150 is constructed such that prediction modes generated by learning in the time series medical data processing device 130 are managed in an integrated manner. The prediction mode database 150 may be implemented in a separate server or storage medium. However, the inventive concept is not limited to this, and the prediction mode may be integrated and managed within the time series medical data processing device 130. The prediction mode may include a time series analysis model for predicting future health conditions by analyzing preprocessed time series medical data. A specific example of such a prediction mode will be described later.

The network 160 may be configured to perform data communication between the terminal 110, the medical database 120, and the time series medical data processing device 130. The terminal 110, the medical database 120, and the time series medical data processing device 130 may exchange data through the network 160 by wire or wirelessly.

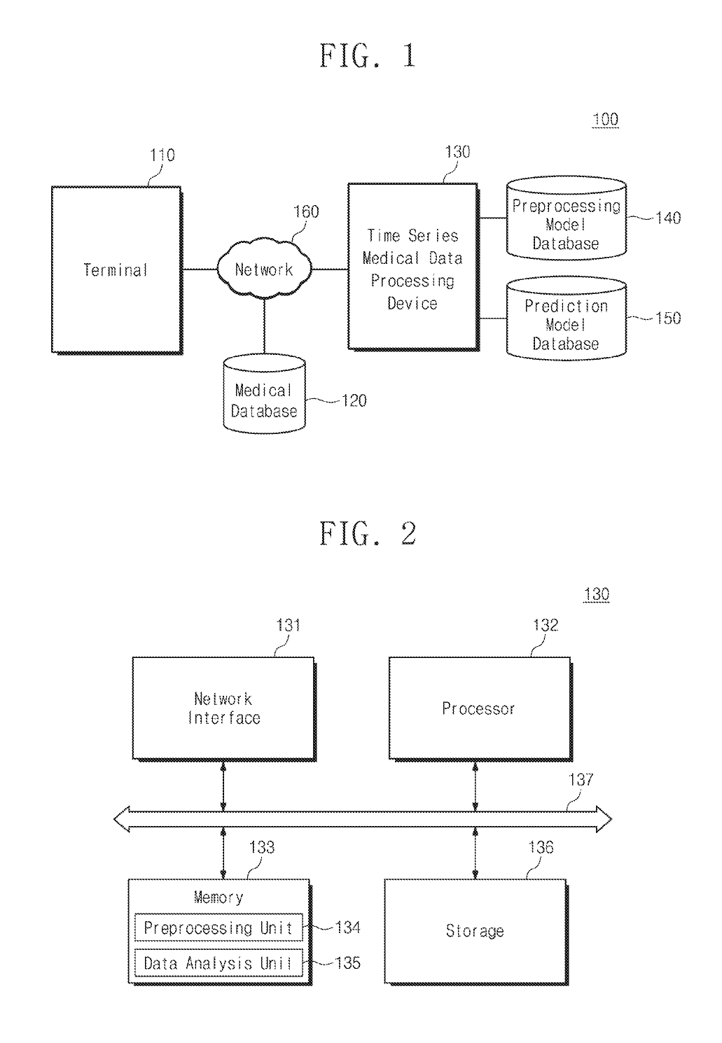

FIG. 2 is an exemplary block diagram of the time series medical data processing device of FIG. 1. The block diagram of FIG. 2 will be understood as an exemplary configuration for preprocessing and analyzing time series medical data, and the structure of the time series medical data processing device will not be limited thereto. Referring to FIG. 2, the time series medical data processing device 130 may include a network interface 131, a processor 132, a memory 133, a storage 136, and a bus 137. Illustratively, the time series medical data processing device 130 may be implemented as a server, but is not limited thereto.

The network interface 131 is configured to receive time series medical data provided from the terminal 110 or the medical database 120 through the network 160 of FIG. 1. The network interface 131 may provide the received time series medical data to the processor 132, the memory 133 or the storage 136 via the bus 137. In addition, the network interface 131 may be configured to provide prediction results of future health conditions generated in response to the received time series medical data to the terminal 110 and the like through the network 160 of FIG. 1.

The processor 132 may function as a central processing device of the time series medical data processing device 130. The processor 132 may perform the control and computational operations required to implement preprocessing and data analysis of the time series medical data processing device 130. For example, according to the control of the processor 132, the network interface 131 may receive time series medical data from the outside. According to the control of the processor 132, a computational operation for generating a learning model may be performed, and future visit data may be calculated using the learning model. The processor 132 may operate utilizing the computation space of the memory 133 and may read files and executable files of the application for running the operating system from the storage 136. The processor 132 may execute the operating system and various applications.

The memory 133 may store data and process codes processed or to be processed by the processor 132. For example, the memory 133 may store time series medical data provided from the network interface 131, information for performing a preprocessing operation, information for computation of future visit data, information for constructing a learning model, and information on the prediction result according to the computation of visit data. The memory 133 may be used as a main memory of the time series medical data processing device 130. The memory 133 may include a dynamic random access memory (DRAM), a static random access memory (SRAM), a phase change RAM (PRAM), a magnetic RAM (MRAM), a ferroelectric RAM (FeRAM), and so on.

The memory 133 may include a preprocessing unit 134 and a data analysis unit 135. The preprocessing unit 134 and the data analysis unit 135 may be part of the computation space of the memory 133. In this case, the preprocessing unit 134 and the data analysis unit 135 may be implemented by firmware or software. For example, the firmware may be stored in the storage 136 and loaded into the memory 133 upon execution of the firmware. Processor 132 may execute firmware loaded into memory 133. The preprocessing unit 134 may preprocess the data under the control of the processor 132 and may operate to build a learning model based thereon. The data analysis unit 135 may analyze the preprocessed data under the control of the processor 132 and may operate to build a learning model based thereon.

Unlike FIG. 2, the preprocessing unit 134 and the data analysis unit 135 may be implemented as separate hardware for preprocessing and analyzing the received time series medical data. For example, the preprocessing unit 134 and the data analysis unit 135 may be implemented in a neuromorphic chip or the like for constructing a learning model by performing teaming through an artificial neural network, or may be implemented in a dedicated logic circuit such as a Field Programmable Gate Array (FPGA) or an Application Specific Integrated Circuit (ASIC).

The preprocessing unit 134 may preprocess the time series medical data. For example, the preprocessing unit 134 may normalize the numerical data of the time series medical data to have the data value in the reference range, and convert the non-numeric data to the numerical data to have the data value in the reference range. The reference range may be a value between 0 and 1. The preprocessing unit 134 may add masking data to the time series medical data to preprocess null data or missing data of the time series medical data to have the specified numerical value. The preprocessing unit 134 may perform preprocessing by reflecting the time-gap data indicating the time interval in the time series medical data. The preprocessing unit 134 may preprocess the dimension of the time series medical data to have a fixed dimension. Based on this preprocessing, a preprocessing model may be learned. Details will be described later.

The data analysis unit 135 may analyze the preprocessed time series medical data, i.e., modeling data. For example, the data analysis unit 135 may analyze the modeling data to predict medical data (visit data) for a future specific time point. The specific time point may be a time point for the health condition that the user wants to know. Based on this data analysis, a prediction mode or time series analysis model may be learned. Details will be described later.

The storage 136 may store data generated by the operating system or applications for the purpose of long-term storage, a file for running the operating system, or executable files of applications. For example, the storage 136 may store files for execution of the preprocessing unit 134 and the data analysis unit 135. The storage 136 may be used as an auxiliary storage device of the time series medical data processing device 130. The storage 136 may include a flash memory, a phase-change RAM (PRAM), a magnetic RAM (MRAM), a ferroelectric RAM (FeRAM), a resistive RAM (RRAM), and so on.

The bus 137 may provide a communication path between the components of the time series medical data processing device 130. The network interface 131, the processor 132, the memory 133, and the storage 136 may exchange data with one another via the bus 137. The bus 137 may be configured to support various types of communication formats used in the time series medical data processing device 130.

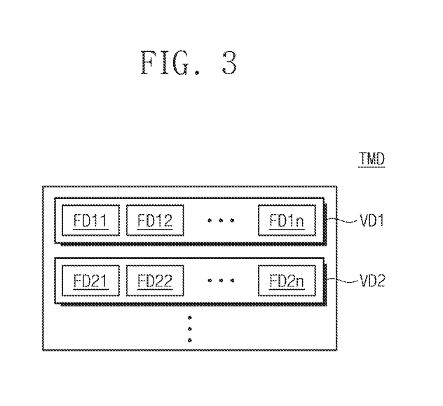

FIG. 3 is a view for explaining time series medical data processed by the time series medical data processing device of FIG. 1. Referring to FIG. 3, time series medical data TMD may include a plurality of visit data. FIG. 3 illustratively shows the time series medical data TMD including first visit data VD1 and second visit data VD2.

Each of the first and second visit data VD1 and VD2, for example, is generated based on diagnosis, treatment, or medication prescriptions, which are provided when the user visits a medical institution such as a hospital. Each of the first and second visit data VD1 and VD2 may be divided according to the visiting turn of the medical institution. For example, the second visit data VD2 may be medical data generated as a result of visiting a medical institution at a particular time in the past. The first visit data VD1 may be medical data generated as a result of visiting the medical institution at a particular time after the second visit data VD2 is generated.

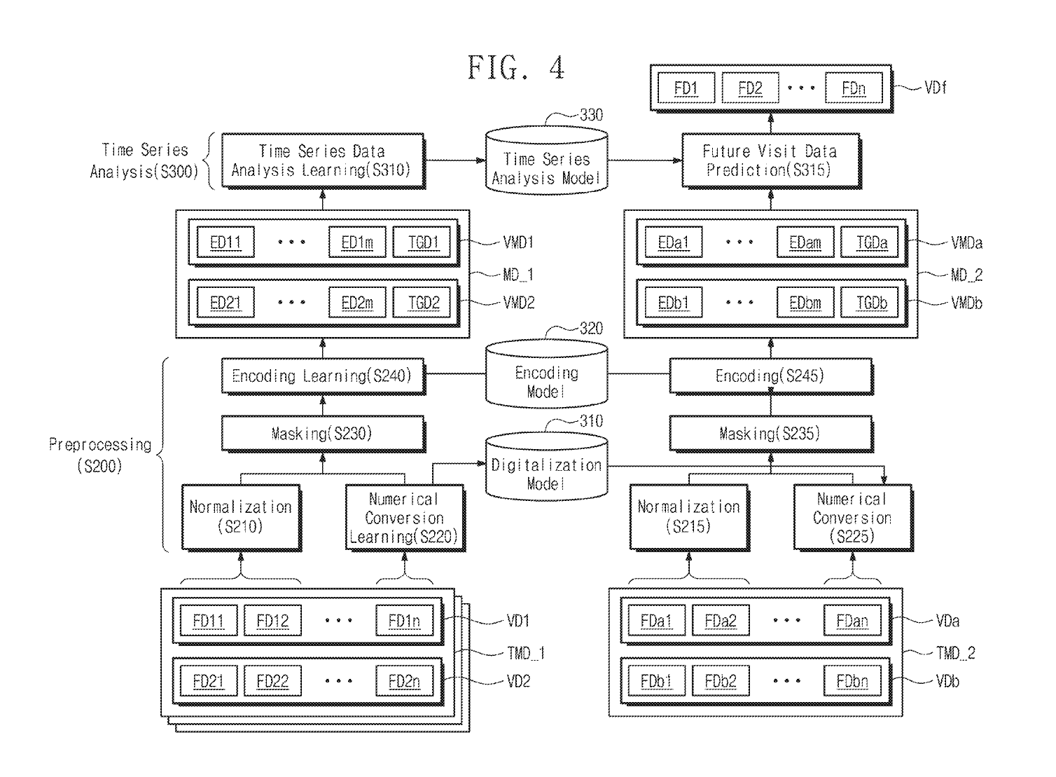

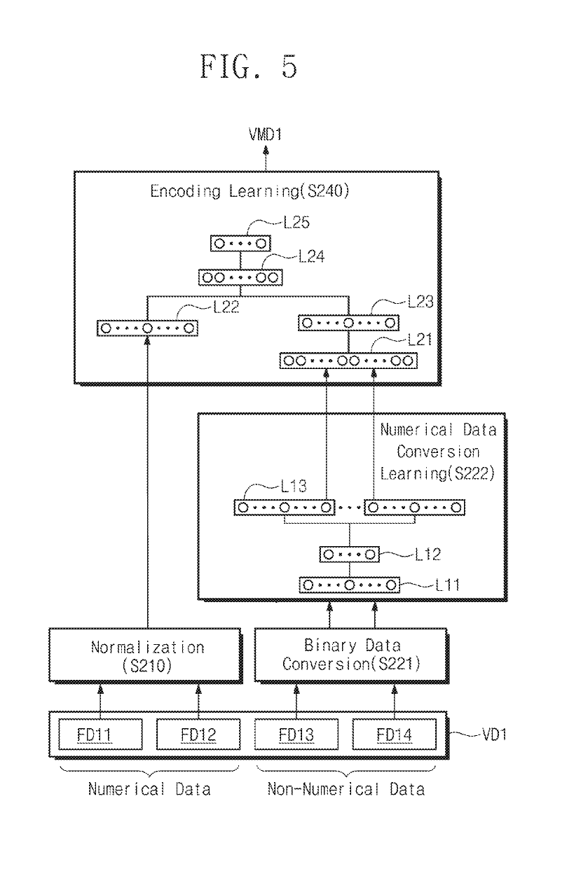

A user's visit to a medical institution may have irregularities. The visit data generated as a result of visiting the medical institution before the first and second visit data VD1 and VD2 may exist, and the time interval of the visit data generated according to the visit result may be irregular. Therefore, time series irregularity of time series medical data TMD may need to be supplemented to ensure accuracy and reliability of health condition prediction. The preprocessing of the time series medical data (TMD) to compensate for this irregularity is illustrated in FIG. 4 and below.

Each of the first and second visit data VD1 and VD2 may include a plurality of feature data. The first visit data may include first to n-th feature data. FD11 to FD1n. The second visit data may include first to n-th feature data FD21 to FD2n. Feature data is generated by personal diagnoses, treatments, or medication prescriptions that are received at a medical facility. For example, the feature data may be disease code data generated based on a specific disease diagnosed according to a user's visit. The feature data may be dosage code data generated based on the prescription of a particular drug. The feature data may be test result data generated based on a specific test result. That is, the time series medical data TMD includes a plurality of visit data according to a visit of a medical institution, and each of a plurality of visit data includes a plurality of feature data generated according to diagnoses, treatments, or prescriptions.

The plurality of feature data may be used for data analysis to ensure accuracy and reliability of health condition prediction. Human future health trends may change based on various variables. Accordingly, the time series medical data processing device 130 of FIG. 1 may preprocess all of the plurality of feature data generated as a result of the visit of the medical institution and reflect them in future health prediction. However, it may be necessary to preprocess multi-dimensional time series medical data TMD in a form that is easy to analyze data in order to secure efficiency of utilizing a plurality of feature data. This preprocessing process is described below with reference to FIG. 4.

Feature data may have various data formats. Feature data, like EMR data, may have a data format that is promised according to a particular disease, prescription, or test, but both numeric and non-numeric data may be mixed. For example, the disease code data generated based on the diagnosis of the disease, and the dosage code data generated based on the drug prescription may include information of a code format such as, for example, E02.31. The test result data generated on the basis of the test result of the body composition, for example, may include information of a numerical format such as blood glucose level, and information of a categorical type (−, +, ++, Etc.) such as hematuria characteristics. Therefore, in order to reflect all of the complex multi-dimensional features in the health condition prediction, supplementation of mixed data formats of time series medical data TMD may be required. The preprocessing of time series medical data TMD to compensate for the diversity of these data types is illustrated in FIG. 4 and below.

The number or types of feature data generated for each visit of the user may be different from each other. The user may not receive the same diagnosis, prescription, or examination at the time of visit of the medical institution. For example, even if a user visits several medical institutions according to the occurrence of a specific disease, a specific diagnosis, prescription, or test may be omitted or added depending on the recovery progress of the user. Therefore, in order to ensure the reliability and efficiency of health condition prediction, it may be necessary to supplement the data sparsity of time series medical data TMD. The preprocessing of the time series medical data (TMD) to compensate for this data sparsity is illustrated in FIG. 4 and below.

FIG. 4 is a view for explaining a data processing process of the time series medical data processing device of FIG. 1. Referring to FIG. 4, the process of processing time series medical data may be classified into operation S200 of preprocessing the time series medical data and operation S300 of analyzing the time series of the preprocessed time series medical data. Each of the operations of FIG. 4 may be performed by the processor 132 of the time series medical data processing device 130 of FIG. 2. Each of the operations of FIG. 4 may be processed by the preprocessing unit 134 and the data analysis unit 135 under the control of the processor 132. For convenience of description, with reference to the reference numerals of FIGS. 1 and 2, FIG. 4 will be described.

Operation S200 of preprocessing the time series medical data includes an operation of generating a preprocessing model using a plurality of time series medical data TMD_1 corresponding to the sample data and an operation of generating personal time series medical data TMD_2. The preprocessing model may include a digitization model 310 and an encoding model 320. The digitization model 310 and the encoding model 320 may be integratedly managed by the preprocessing model database 140 of FIG. 1. A plurality of time series medical data TMD_1 may be provided from the medical database 120 of FIG. 1 and personal time series medical data TMD_2 may be provided from the terminal 110 of FIG. 1.

In the operation of generating a preprocessing model using a plurality of time series medical data TMD_1 (hereinafter referred to as time series medical data), operation S210 of normalizing the time series medical data TMD_1, operation S220 of learning numerical conversion, operation S230 of masking, and operation S240 of learning encoding may be performed. Operations S210 to S240 may be changed in time sequence, unlike that shown in FIG. 4. For example, operations S210 and S220 may be performed after operation S230 is performed first.

As described in FIG. 3, the time series medical data TMD_1 may include first and second visit data VD1 and VD2. The first visit data VD1 may be generated by visiting the medical institution for a first time. The second visit data VD2 may be generated by visiting the medical institution for a second time before the first time. Although not shown in the drawing, visit data generated by visiting a medical institution for a time before the second time may be further included in the time series medical data TMD_1. The first visit data VD1 includes a plurality of feature data FD11 to FD1n, and the second visit data VD2 includes a plurality of feature data FD21 to FD2n. Hereinafter, for convenience of explanation, operation S200 will be described based on a plurality of feature data FD11 to FD1n included in the first visit data VD1.

In operation S210, numerical data among a plurality of feature data FD11 to FD1n may be normalized. Illustratively, the first and second feature data FD11 and FD12 are described as numerical data. Each of the first and second feature data FD11 and FD12 may have a numerical value in an independent range according to tested features. Under the control of the processor 132, the preprocessing unit 134 may normalize each of the first and second feature data FD11 and FD12 to have a data value in the reference range. For example, the reference range may have a value between 0 and 1.

In operation S220, a digitalization model 310 for converting non-numeric data among a plurality of feature data. FD11 to FD1n into numerical data may be generated. Illustratively, the n-th feature data FD1n is described as non-numeric data, such as a code or categorical type. In operation S220, under the control of the processor 132, the n-th feature data FD1n may be converted into numerical data. Under the control of the processor 132, the digitization model 310 may be learned based on conversion into numerical data. The learned digitization model 310 may be updated in the preprocessing unit 134. The digitization model 310 may be integrally managed in the preprocessing model database 140 of FIG. 1 and may be constructed, for example, in the storage 136 of FIG. 2. However, the inventive concept is not limited thereto, and the digitalization model 310 may be constructed on a separate server or storage medium.

In operation S220, under the control of the processor 132, the preprocessing unit 134 may convert the n-th feature data FD1n into a numerical vector composed of binary data such as 0 and 1 and convert the converted numerical vector to have the data value in the reference range again. That is, all of the first to n-th feature data FD11 to FD1n may have a data value in the reference range. Therefore, the time series medical data (TMD_1), in which the numerical data and the non-numerical data are mixed, may be preprocessed as the uniform numerical data so that the complex feature data may be reflected in the prediction of the future health condition.

In operation S230, masking data may be added to the digitized time series medical data. As described with reference to FIG. 3, the user may not receive the same test at each visit of the medical institution. Feature data for unchecked features may appear as null or missing data. The masking data may be configured to distinguish feature data having a data value from feature data having a missing data value. For example, the masking data may include first through n-th feature masking data. Feature masking data corresponding to feature data having a data value may have a first data value (e.g., 1). Feature masking data corresponding to feature data having a missing data value may have a second data value (e.g., 0).

In operation S230, under the control of the processor 132, the preprocessing unit 134 may encode the time series medical data and the masking data together. For example, the processor 132 may use masking data to replace the missing data value with a second data value (e.g., 0) and may perform preprocessing for encoding using the second data value. Thus, the error of the integrated encoding by the missing data value may be minimized.

In operation S240, the digitized and masked time series medical data may be generated as the encoding model 320 for encoding it as modeling data MD_1. The modeling data MD_1 may include first modeling visit data VMD_1 and second modeling visit data VMD_2. The first modeling visit data VMD_1 may include first through m-th encoded data ED11 to ED1m. The second modeling visit data VMD_2 may include first through m-th encoded data ED21 to ED2m. m may be a natural number smaller than n, but is not limited thereto. That is, time series medical data TMD_1 may be preprocessed as modeling data MD_1 having reference dimensions. For example, the dimension of time series medical data may be reduced.

In operation S240, under the control of the processor 132, the preprocessing unit 134 may convert the time series medical data TMD_1 into modeling data MD_1, and based on this conversion, the encoding model 320 may be learned. The learned encoding model 320 may be updated by the preprocessing unit 134 of FIG. 2. The encoding model 320 may be integrally managed in the preprocessing model database 140 of FIG. 1 and may be constructed, for example, in the storage 136 of FIG. 2. However, the inventive concept is not limited thereto, and the encoding model 320 may be constructed on a separate server or storage medium.

The modeling data MD_1 may further include first time-gap data TGD1 and second time-gap data TGD2. The first time-gap data TGD1 may be included in the first modeling visit data VMD_1. The first time-gap data TGD1 may be generated based on a difference between a first time at which the first visit data VD1 is generated and a second time at which the second visit data VD2 is generated. The second time-gap data TGD2 may be included in the second modeling visit data VMD_2. The second time-gap data TGD2 may be generated based on the difference between the second time and the visit time before the second time. Since the first and second time-gap data TGD1 and TGD2 are reflected in the modeling data MD_1, time series irregularities in medical data may be solved and the accuracy and reliability of prediction of future health condition may be secured.

Although FIG. 4 shows that the modeling data MD_1 includes the first and second time-gap data TGD1 and TGD2, this is not limited thereto. For example, before operation S240 is performed, the first and second time-gap data TGD1 and TGD2 may be reflected. In this case, the first through m-th encoded data ED11 to ED1m may include a component to which the first time-gap data TGD1 is reflected.

The first and second time-gap data TGD1 and TGD2 may be converted into units of day, month, year and the like and may be digitized. For example, if the difference between the first and second time is one year and one month, the time-gap information may be numerically expressed as 395 in a day, 13 in a month, 1.083 in a year, and so on. This digitized time-gap information may be converted to a data value having a reference range (e.g., between 0 and 1) to generate the first time-gap data TGD1. Under the control of the processor 132, the preprocessing unit 134 digitizes the difference between the first and second times, and converts it to a data value having a reference range to generate the first and second time-gap data TGD1 and TGD2.

In the operation of preprocessing personal time series medical data TMD_2, operation S215 of normalizing the numerical data among the personal time series medical data TMD_2, operation S225 of numerically converting non-numeric data among the personal time series medical data TMD_2, and operation S235 of masking, and operation S245 of encoding may be performed. The personal time series medical data TMD_2 may include first and second personal visit data VDa and VDb. The first personal visit data VDa includes a plurality of feature data FDa1 to FDan, and the second personal visit data VDb includes a plurality of feature data FDb1 to FDbn.

In operation S215, the numerical data in the personal time series medical data TMD_2 may be normalized to have the data value in the reference range. Operation S215 may be substantially the same as operation S210.

In operation S225, the non-numeric data of the personal time series medical data TMD_2 may be converted to have the data value in the reference range. Under the control of the processor 132, the preprocessing unit 134 may convert the non-numeric data into numeric data based on the digitization model 310 constructed in operation S220.

In operation S235, masking data may be added to the digitized personal time series medical data. Operation S235 may be substantially the same as operation S230.

In operation S245, digitized and masked time series medical data may be encoded to personal modeling data MD_2. Under the control of the processor 132, the preprocessing unit 134 may generate personal modeling data MD_2 based on the encoding model 320 constructed in operation S240. As described in the modeling data MD_1 generation process, the time-gap data TGDa and TGDb may also be reflected in the personal modeling data MD_2. The time-gap data TGDa and TGDb may be included in the personal modeling data MD_2. Alternatively, the components of the time-gap data TGDa and TGDb may be reflected in each of a plurality of feature data FDa1 to FDan and FDb1 to FDbn.

Operation S300 of analyzing the time series for the preprocessed time series medical data may include operation S310 of learning by analyzing the time series data using the modeling data MD_1, and operation S315 of predicting future visit data using the time series analysis model 330 generated through learning. The time series analysis model 330 may be integratedly managed by the prediction mode database 150 of FIG. 1.

In operation S310, the time series data modeling data MD_1 may be analyzed and the time series analysis model 330 may be generated based on this analysis. The time series analysis model 330 may be implemented as a circular neural network of a Long-Short Term Memory (LSTM) scheme, for example. Under the control of the processor 132, the data analysis unit 135 may analyze the modeling data MD_1 to calculate future visit data by time series medical data TMD_1. Future visit data may be predicted visit data expected at a specified future time point, based on the time series trend of the time series medical data TMD_1. Under the control of the processor 132, the data analysis unit 135 may repeat the calculation of future visit data to learn the time series analysis model 330. The time series analysis model 330 is learned to comprehensively consider the relationship between the plurality of feature data FDa1 to FDan and FDb1 to FDbn in addition to the individual data values of the plurality of feature data FDa1 to FDan and FDb1 to FDbn included in the first and second personal visit data VDa and VDb. The learned time series analysis model 330 may be updated by the data analysis unit 135 of FIG. 2. The time series analysis model 330 may be constructed in the storage 136 of FIG. 2, but may be constructed in a separate server or storage medium.

In operation S315, future visit data VDf for a future specific time point that the user wants to know may be predicted based on personal modeling data MD_2. Under the control of the processor 132, the data analysis unit 135 may generate the future visit data VDf based on the time series analysis model 330 constructed in operation S310. The future visit data VDf may include a plurality of feature data FD1 to FDn. The dimension of the future visit data VDf may be equal to the dimension of the first personal visit data VDa and the second personal visit data VDb. Since the plurality of feature data FD1 to FDn collectively consider a relation between the plurality of feature data FDa1 to FDan and FDb1 to FDbn in addition to the individual data values of the plurality of feature data FDa1 to FDan and FDb1 to FDbn included in the first and second personal visit data VDa and VDb, the reliability and accuracy of future health conditions may be ensured.

FIG. 5 is a view for explaining a preprocessing process in the method of processing time series medical data of FIG. 4. Referring to FIG. 4, the first visit data VD1 is preprocessed through operations S210 to S240. The first visit data VD1 illustratively includes first to fourth feature data FD11 to FD14. The first and second feature data FD11 and FD12 are assumed to be numeric data, and the third and fourth feature data FD13 and FD14 are assumed to be non-numeric data. For convenience of explanation, operation S230 of FIG. 4 is omitted. Referring to the reference numerals of FIGS. 2 and 4, FIG. 5 will be described.

In operation S210, the first and second feature data FD11 and FD12 are normalized to a data value having a reference range. Operation S210 is substantially the same as operation S210 in FIG. 4, so a detailed description thereof will be omitted.

Operation S221 and operation S222 correspond to operation S220 in FIG. 4. In operation S221, the third and fourth feature data FD13 and FD14 may be converted into a numerical vector composed of binary data. Illustratively, under the control of the processor 132 of FIG. 2, the preprocessing unit 134 uses the one-hot encoding or the multi-hot encoding to convert the third and fourth feature data FD13 and FD14 into an array of logic values of 0 and logic values of 1.

In operation S222, the third and fourth feature data converted into the numerical vector may be converted to have the data value in the reference range. Under the control of the processor 132, the preprocessing unit 134 may convert the non-numeric data into numeric data based on the digitization model 310 constructed in operation S220. Also, the digitization model 310 may be learned and updated through the conversion process of the third and fourth feature data FD13 and FD14. Illustratively, in operation S222, under the control of the processor 132, the preprocessing unit 134 may digitize the third feature data FD13 and the fourth feature data FD14 in Word2Vec manner.

In operation S222, the third and fourth feature data converted into the numerical vector may output the data value in the reference range through the first to third layers L11 to L13 of the digitalization model 310. Through the first to third layers L11 to L13, as the data values of the third and fourth feature data FD13 and FD14 and also the association between the third feature data FD13 and the fourth feature data FD14 are reflected, the output data may be determined. For example, when two non-numeric data (third and fourth feature data FD13 and FD14) are digitized, the output data by the digitalization model 310 may include two-dimensional data corresponding to the third feature data FD13 and two-dimensional data corresponding to the fourth feature data FD14.

In operation S240, the first to fourth normalized or numerically converted feature data may be converted into first modeling data VMD1 having a predetermined dimension. Operation S240 corresponds to operation S240 in FIG. 4. Under the control of the processor 132, the preprocessing unit 134 may execute the constructed encoding model 320 to generate the first modeling data VMD1. Moreover, under the control of the processor 132, the preprocessing unit 134 may learn and update the encoding model 320 through the process of generating the first modeling data VMD1.

In operation S240, the normalized or numerically converted first to fourth feature data may output fixed-dimensional data values through the first to fifth layers L21 to L25 of the encoding model 240. Through the first to fifth layers L21 to L25, as the data values of the first to fourth feature data FD11 and FD14 and also the association between the first to fourth feature data FD11 and FD14 are reflected, the output data may be determined. In operation S222, the two-dimensional data corresponding to the third and fourth feature data FD13 and FD14 may be reduced to one-dimensional data through the first layer L21. One-dimensional data corresponding to the third and fourth feature data FD13 and FD14 and one-dimensional data by normalization of the first and second feature data FD11 and FD12 may be integrated through the second to fourth layers L22 to L24, and may be outputted as the first modeling data VMD1 having a fixed dimension through the fifth layer L25.

In summary, by converting the first visit data VD1 in which numeric data and non-numeric data are mixed into a digitalized form having a reference range, the speed and efficiency of data analysis may be ensured in the future. In addition, by considering and analyzing various aspects of time series medical data in a complex way, accuracy and reliability of future visit data may be ensured.

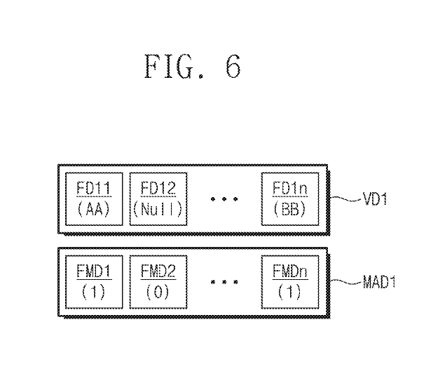

FIG. 6 is a view for explaining an application process of masking data in the method of processing time series medical data of FIG. 4. Referring to FIG. 6, the first visit data VD1 includes first to n-th feature data FD11 to FD1n. The first masking data MAD1 includes first to n-th feature masking data FMD1 to FMDn. The number of feature data and the number of feature masking data may be the same. The first to n-th feature masking data FMD1 to FMDn correspond to the first to n-th feature data FD11 to FD1n, respectively.

In the first visit data VD1, the first feature data FD11 has a data value of AA, the second feature data FD12 has a null data value, and the n-th feature data FD1n has a data value of BB. The data value of AA and the data value of BB may be digitalized data values, but are not limited thereto. At the time of generation of the first visit data VD1, the test or prescription corresponding to the second feature data FD12 may not proceed. In this case, the modeling data generated in the processing of the second feature data FD12 of FIGS. 4 and 5 may cause an error of future visit data or may cause an incorrect prediction result.

The first masking data MAD1 is configured to distinguish null data in the first visit data VD1. That is, the first masking data MAD1 may be configured to distinguish between the inspected feature and the unchecked feature at the time of generating the first visit data VD1. For example, the first feature masking data FMD1 and the n-th feature masking data FMDn may have a first data value. The first data value may be one. The second feature masking data FMD2 may have a second data value. The second data value may be one. That is, the second feature data FD12 having the null data and the remaining feature data may be distinguished through the first masking data MAD1.

In the preprocessing process, the data value of the second feature data FD12 may be replaced with 0, which is the data value of the second feature masking data FMD2. For this, a multiplication computation may be performed between the first visit data VD1 and the first masking data MAD1. That is, the data values of the first feature data FD11 and the n-th feature data FD1n multiplied by 1 are maintained, and the data value of the second feature data FD12 multiplied by 0 may be replaced with zero. Thus, errors in future visit data caused by null data (missing data) may be minimized. However, the inventive concept is not limited to this, and the data values of the second feature data FD12 may be replaced with other values in various ways.

For example, in the preprocessing process, visit data (previous visit data) according to the previous visit of the first visit data VD1 and visit data (next visit data) following the next visit of the first visit data VD1 may exist. And, feature data corresponding to the second feature data FD12 may exist in the previous visit data, and thereafter, the feature data corresponding to the second feature data FD12 may exist in the visit data. In this case, the data value of the second feature data FD12 may be replaced with an intermediate value of feature data corresponding to the second feature data FD12 in the previous visit data and feature data corresponding to the second feature data FD12 in the following visit data.

For example, in the preprocessing process, visit data (previous visit data) according to the previous visit of the first visit data VD1 may exist. Then, in the previous visit data, feature data corresponding to the second feature data FD12 may exist. In this case, the data value of the second feature data FD12 may be replaced with the feature data corresponding to the second feature data FD12 in the previous visit data.

For example, in the preprocessing process, a plurality of visit data according to previous or following visits of the first visit data VD1 may exist. Then, in the plurality of visit data, a plurality of feature data corresponding to the second feature data FD12 may exist. In this case, the data value of the second feature data FD12 may be replaced with the average value of all feature data corresponding to the second feature data FD12.

A device and method for processing multi-dimensional time series medical data according to an embodiment of the inventive concept enables modeling of time series medical data to have a fixed dimension, thereby enabling the prediction of health condition utilizing human complex features.

Also, a device and method for processing multi-dimensional time series medical data according to an embodiment of the inventive concept may ensure the efficiency of future health condition prediction by preprocessing time series medical data through masking, time-gap, and digitalization, or building a learning model for preprocessing.

Although the exemplary embodiments of the inventive concept have been described, it is understood that the inventive concept should not be limited to these exemplary embodiments but various changes and modifications can be made by one ordinary skilled in the art within the spirit and scope of the inventive concept as hereinafter claimed.

What is claimed is:

1. A device for processing multi-dimensional time series medical data, the device comprising:
a network interface configured to receive time series medical data including first visit data corresponding to a first time and second visit data corresponding to a second time before the first time;
a preprocessing unit configured to preprocess the time series medical data to generate modeling data;
a data analysis unit configured to generate a time series analysis model for predicting future visit data corresponding to a third time after the first time from the modeling data; and
a processor configured to control the preprocessing unit and the data analysis unit,
wherein the preprocessing unit is configured to preprocess the first visit data based on a difference between the first time and the second time.

2. The device of claim 1, wherein the modeling data comprises first modeling visit data obtained by preprocessing the first visit data, and second modeling visit data obtained by preprocessing the second visit data,
wherein the first modeling visit data comprises time-gap data generated based on a difference between the first time and the second time.

3. The device of claim 1, wherein the preprocessing unit performs preprocessing to change a dimension of each of the first visit data and the second visit data to a reference dimension based on an encoding model.

4. The device of claim 1, wherein the preprocessing unit generates an encoding model for changing a dimension of each of the first visit data and the second visit data to a reference dimension.

5. The device of claim 1, wherein the first visit data comprises first feature data that is numeric data and second feature data that is non-numeric data,
wherein the preprocessing unit is configured to convert the second feature data into numerical data.

6. The device of claim 5, wherein the preprocessing unit is configured to normalize the first feature data to have a numerical value in a reference range, convert the non-numeric data of the second feature data into binary data, and convert the binary data into numerical data having a numerical value in the reference range based on a digitalization model.

7. The device of claim 5, wherein the preprocessing unit is configured to generate a digitalization model for converting the second feature data into numerical data.

8. The device of claim 1, wherein the preprocessing unit is configured to generate first masking data having a first data value when target feature data exists in the first visit data and a second data value different from the first data value when the target feature data does not exist in the first visit data, and generate second masking data having the first data value when the target feature data exists in the second visit data and the second data value when the target feature data does not exist in the second visit data.

9. The device of claim 8, wherein the preprocessing unit is configured to generate first modeling visit data by preprocessing the first visit data and the first masking data, and generate second modeling visit data by preprocessing the second visit data and the second masking data.

10. The device of claim 8, wherein the preprocessing unit is configured to add the target feature data having the second data value to the first visit data or the second visit data when the target feature data does not exist in the first visit data or the second visit data.

11. A method for processing multi-dimensional time series medical data by a processor, the method comprising:
preprocessing a first visit data including a plurality of feature data extracted during a first time and a second visit data including a plurality of feature data extracted during a second time before the first time; and
learning a time series analysis model for predicting future visit data including a plurality of feature data based on the preprocessed first and second visit data,
wherein the preprocessing of the first visit data and the second visit data comprises preprocessing the first visit data by reflecting time-gap data corresponding to a difference between the first time and the second time in the first visit data.

12. The method of claim 11, wherein the preprocessing of the first visit data and the second visit data further comprises learning an encoding model for changing a dimension of each of the first and second visit data to a reference dimension based on the first and second visit data.

13. The method of claim 12, further comprising:
preprocessing personal time series medical data based on the learned encoding model; and
predicting personal future visit data based on the preprocessed personal time series medical data and the learned time series analysis model.

14. The method of claim 12, wherein the preprocessing of the first visit data and the second visit data further comprises:
adding first masking data to the first visit data; and
adding second masking data having the same dimension as the first masking data to the second visit data,
wherein the first masking data comprises first feature masking data, and a data value of the first feature masking data is determined based on whether feature data corresponding to the first feature masking data exist among the plurality of feature data included in the first visit data,
wherein the second masking data comprises second feature masking data, and a data value of the second feature masking data is determined based on whether feature data corresponding to the second feature masking data exists among the plurality of feature data included in the second visit data,
wherein the encoding model is learned based on the first and second visit data and the first and second masking data.

15. The method of claim 11, wherein the preprocessing of the first visit data and the second visit data further comprises learning a digitalization model for converting non-numeric data into numeric data having a data value in a reference range based on the non-numeric data among a plurality of feature data included in the first and second visit data.

16. The method of claim 15, wherein the preprocessing of the first visit data and the second visit data further comprises:
normalizing numerical data among the plurality of feature data included in the first and second visit data to have a data value in the reference range; and
learning an encoding model for changing a dimension of each of the first and second visit data to a reference dimension based on the first and second visit data normalized or converted to have the data value in the reference range.

17. The method of claim 16, further comprising:
normalizing numerical data included in personal time series medical data to have the data value in the reference range;
converting non-numeric data included in the personal time series medical data into numerical data having the data value in the reference range based on the learned digitalization model; and
changing a dimension of the normalized or converted personal time series medical data to a reference dimension based on the learned encoding model.
